(19) United States
(12) Patent Application Publication
(10) Pub. No.: US 2006/0195036 A1
(43) Pub. Date: Aug. 31, 2006

(54) Heartbeat measuring device

(76) Inventor: Hisao Nakamura, Chiba-shi (JP)

Correspondence Address: BRUCE L. ADAMS, ESQ.;SUITE 1231, 17 BATTERY PLACE, NEW YORK, NY, 10004, US

(21) Appl. No.: 11/360,866

(22) Filed: Feb. 23, 2006

(30) Foreign Application Priority Data
Feb. 25, 2005 (JP) 2005-051305

(51) Int. Cl. A61B 5/0402

(52) U.S. Cl. 600/509

(57) ABSTRACT

To provide a heartbeat measuring device in which a ferrite antenna is accommodated in and fixed to a case without using a complicated fixing structure and while being adapted so as not to undergo an influence of an external impact. A transmitting ferrite antenna of a heart rate transmitter of the heartbeat measuring device is one constituted with a winding being applied to a rod-like ferrite core. In a heart rate transmitter case, there is formed an antenna accommodation concave part. The transmitting ferrite antenna is held in the antenna accommodation concave part with a cap-like antenna frame mounted to both ends of a rod-like ferrite core being fitted to an antenna frame fitting part of the antenna accommodation concave part. Further, an adhesive having an elasticity is filled in the antenna accommodation concave part.

### BACKGROUND OF THE INVENTION

1. Field of the Invention

The present invention relates to a heartbeat measuring device. Especially, it is one relating to an antenna fixation of the heartbeat measuring device.

2. Description of the Prior Art

As disclosed in JP-A-10-155753 Gazette for instance, the heartbeat measuring device for measuring a heart rate during a movement is constituted by a heart rate transmitter mounted to a chest of a user and a heart rate receiver mounted to an arm of the user. And, the heart rate transmitter is constituted by one pair of chest electrodes, a heart rate operation section for outputting a heart rate data on the basis of an electrocardiosignal from the chest electrodes, a data transmission section for converting the heart rate data into a radio signal, a transmitting antenna for transmitting the radio signal, an electric source battery, and a chest-mounted type case for accommodating these components. The chest-mounted type case is constituted by a transmitter case and a chest belt. Further the heart rate receiver is constituted while containing a reception antenna for receiving the radio signal, a signal processing section for processing the radio signal, and a display part for displaying the heart rate and the like.

By the way, the transmission antenna is a ferrite antenna in which a winding has been applied to a ferrite core. Generally, the ferrite antenna is weak against an impact and, for this reason, it breaks if directly undergoes the impact from a transmitter case.

Whereupon, in the heartbeat measuring device mentioned above, the transmitting ferrite antenna is attached to the transmitter case by an antenna connection plate having a spring part so as to be hung while providing an interstice. Since it is an antenna fixation structure like this, the impact that the transmitter case has undergone is absorbed in the spring part of the antenna connection plate, and it follows that the impact is not applied directly to the transmitting ferrite antenna, thereby contriving an improvement in impact resistance. However, the antenna connection plate, having the spring part, that is an antenna fixation member is very complicated in its structure, and accordingly there is a problem that its manufacture takes a lot of labor hour and trouble and brings about an increase in cost. Further, there is also a problem that, when a large impact has been applied to the case, the spring part of the antenna connection plate extremely deforms, so that the antenna connection plate contacts with an inner wall of the case.

A problem that the present invention is to solve is to provide a heartbeat measuring device in which the ferrite antenna is accommodated in and fixed to the case without using a complicated fixing structure and while being adapted so as not to undergo an influence of an external impact.

### SUMMARY OF THE INVENTION

A heartbeat measuring device of the present invention is characterized in that a ferrite antenna has been accommodated in an antenna accommodation concave part of a case through a cap-like antenna frame having an elasticity. And, the antenna frame is a cap-like antenna frame mounted to both ends of a rod-like ferrite core of the ferrite antenna. Further, there maybe adapted such that the ferrite antenna in which the cap-like antenna frame has been mounted to both ends is held in the antenna accommodation concave part with the cap-like antenna frame being fitted respectively to both ends of the antenna accommodation concave part of the case, or the ferrite antenna in which the cap-like antenna frame has been mounted to both ends is held in the antenna accommodation concave part with the cap-like antenna frame being pressed by a convex part formed in a case back of the case. An adhesive having the elasticity is filled in an interstice between the antenna accommodation concave part and the ferrite antenna accommodated in the former.

### BRIEF DESCRIPTION OF THE SEVERAL VIEWS OF THE DRAWINGS

A preferred form of the present invention is illustrated in the accompanying drawings in which:

FIG. 1 is a plan view of a heart rate transmitter of an embodiment 1 of the present invention, shown with a case back and electronic components being removed;

FIG. 2 is a perspective view of the heart rate transmitter of the embodiment 1 of the present invention, shown with the case back being removed;

FIG. 3 is a sectional view of the heart rate transmitter of the embodiment 1 of the present invention, cut in a line A-A of the plan view of FIG. 1; and

FIG. 4 is a sectional view of the heart rate transmitter of the embodiment 1 of the present invention, cut in a line B-B of the plan view of FIG. 1.

### DETAILED DESCRIPTION OF THE PREFERRED EMBODIMENTS

The present invention is a heartbeat measuring device characterized in that a ferrite antenna has been accommodated in an antenna accommodation concave part of a case through a cap-like antenna frame having an elasticity. The cap-like antenna frame is fitted respectively to both ends of the antenna accommodation concave part of the case. By doing like this, the ferrite antenna having been accommodated in the antenna accommodation concave part and held in the case is prevented from the impact applied to the case.

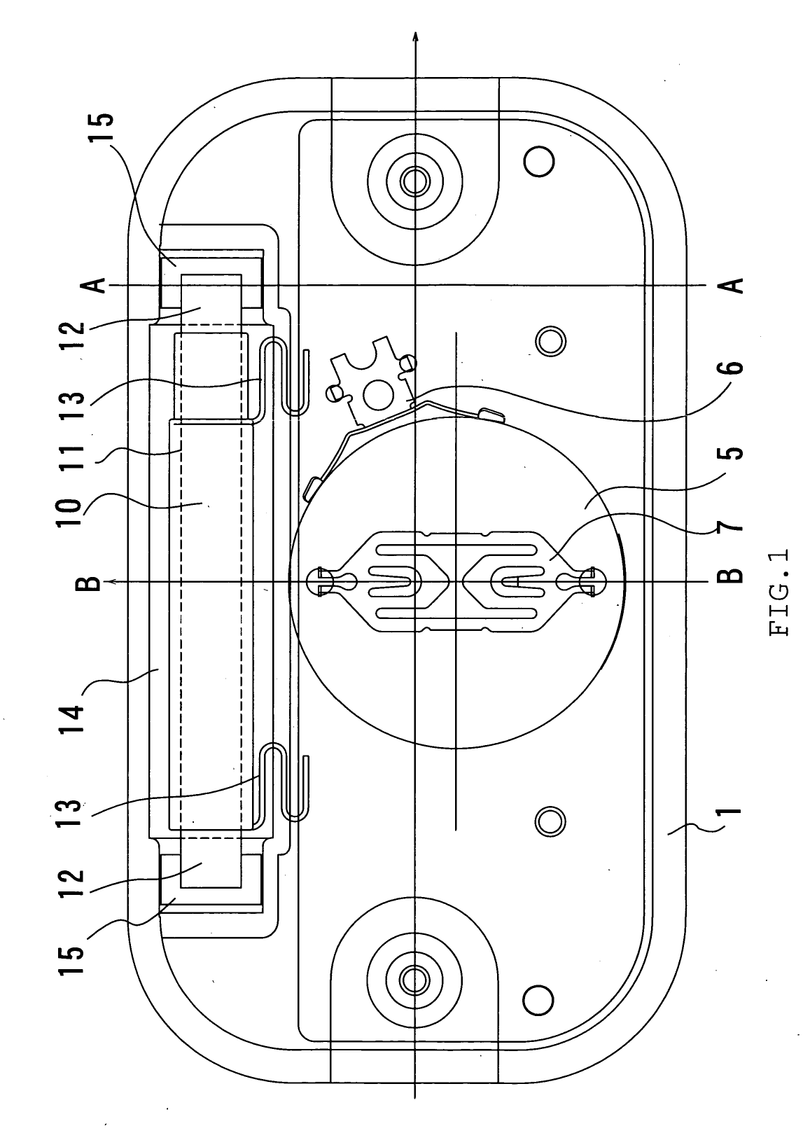

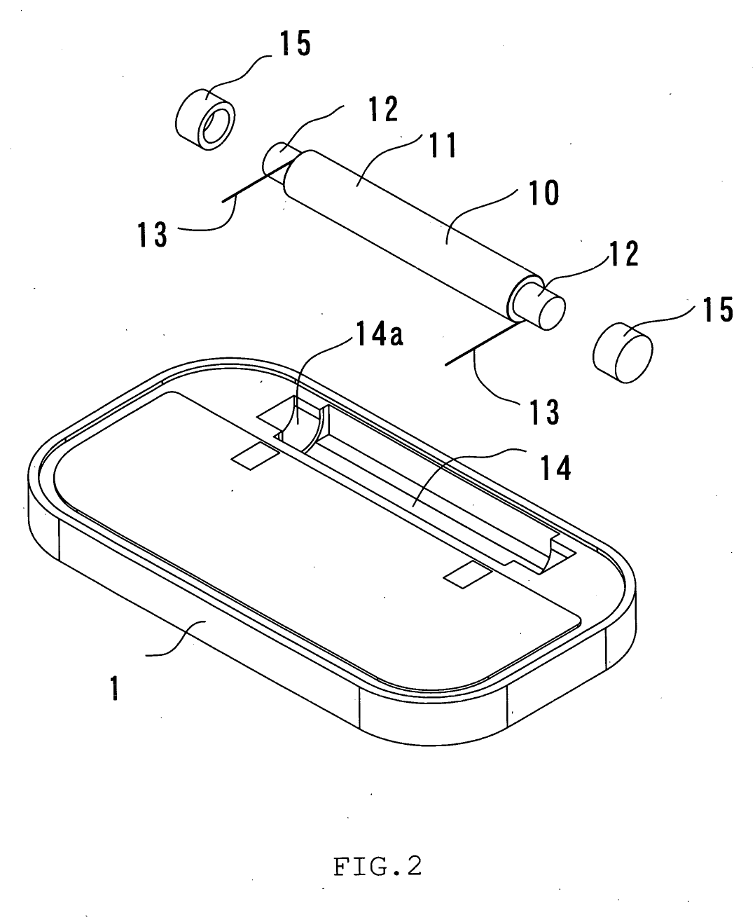

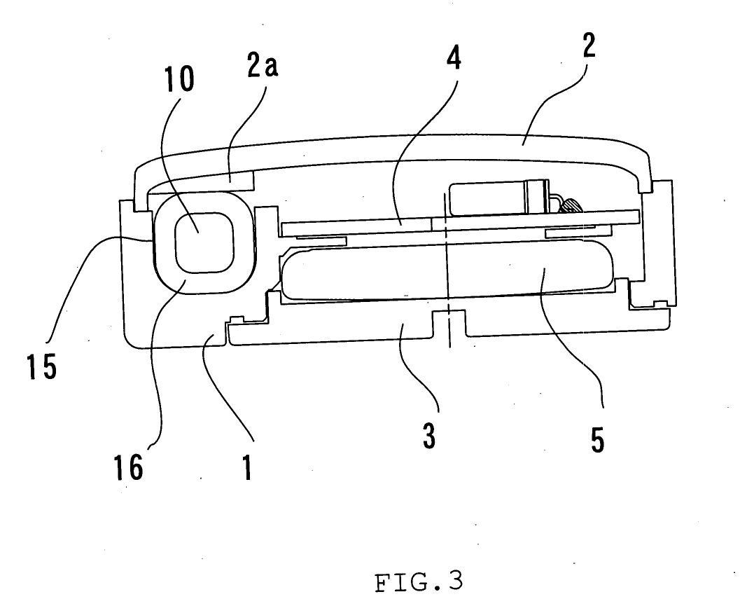

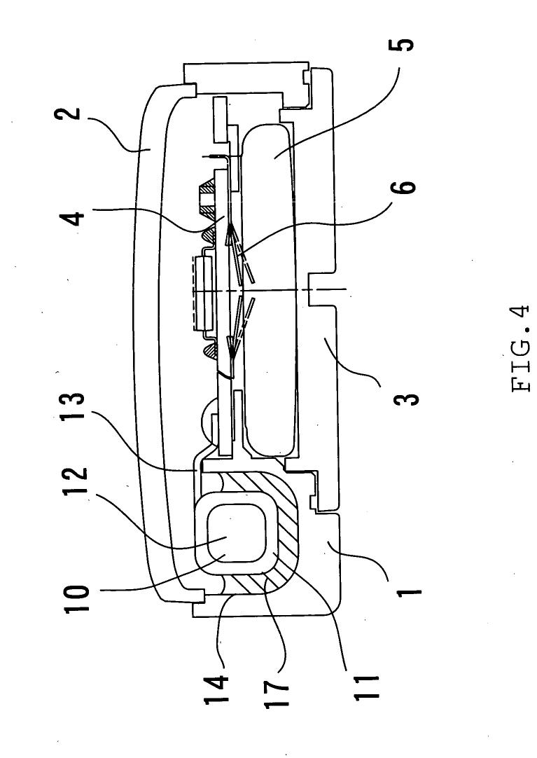

An embodiment 1 of the present invention is shown in a plan view of FIG. 1 shown with a case back being removed, a perspective view of FIG. 2 shown with the case back and electronic components being removed, a sectional view of FIG. 3 cut in a line A-A of the plan view, and a sectional view of FIG. 4 cut in a line B-B of the plan view. Within the heartbeat measuring device, it is one in which, in the heart rate transmitter mounted to the chest of the user, a transmitting ferrite antenna 10 has been accommodated in an antenna accommodation concave part 14 of a transmitter case 1 through an antenna frame 16 having the elasticity.

The heart rate transmitter is constituted by one pair of chest electrodes, the heart rate operation section for outputting the heart rate data on the basis of the electrocardiosignal from the chest electrodes, the data transmission section for converting the heart rate data into the radio signal, the transmitting antenna for transmitting the radio signal, the electric source battery, and the chest-mounted type case for accommodating these components. This is a basic constitution of the general heart rate transmitter.

In the embodiment 1, the chest-mounted type case is an approximately rectangular transmitter case 1, and the electric source battery is a battery 5. The transmitter case 1 possesses a case back 2 and a battery lid 3. Further, the heart rate operation section and the data transmission section are realized by a circuit board 4 to which electronic components such as IC are mounted. The transmitting ferrite antenna 10 is one constituted with a winding 11 being applied to a rod-like ferrite core 12. The transmitting ferrite antenna 10 is connected to the circuit board 4 by a lead wire 13.

As shown in FIG. 1 and FIG. 2, in the approximately rectangular transmitter case 1 there is formed in its center part a battery accommodation concave part, and in a circumference of this battery accommodation concave part there is disposed a plus terminal plate 6 having a spring property and in a bottom face of the same a minus terminal plate 7 having the spring property, respectively. If the battery 5 is accommodated in the battery accommodation concave part, the plus terminal plate 6 and the minus terminal plate 7 butt respectively against a plus electrode and a minus electrode of the battery 5.

The antenna accommodation concave part 14 is provided between a side face of the approximately rectangular transmitter case 1 and the battery accommodation concave part. The antenna accommodation concave part 14 is a groove-like concave part having a shape and a dimension, which correspond to a size of the ferrite antenna 10 to be accommodated. That is, the antenna accommodation concave part 14 is a groove of a shape and a dimension, by which to its both ends there is fitted a cap-like antenna frame mentioned later, and is the groove in which its intermediate part accommodates the winding 11 of the ferrite antenna 10 so as not to contact with a wall face. The antenna accommodation concave part 14 and the plastic-made transmitter case 1 possessing the battery accommodation concave part mentioned above are manufactured by a plastics molding.

The antenna frame 16 is a cap-like component, having the elasticity, of a dimension by which the rod-like ferrite core 12 of the transmitting ferrite antenna 10 is fitted. The antenna frame 16 is manufactured by molding an elastic material such as silicone material like a cap.

The transmitting ferrite antenna 10 in which the winding 11 has been applied to the rod-like ferrite core 12 is accommodated into the antenna accommodation concave part 14 of the transmitter case 1 after the cap-like antenna frame 16 having the elasticity has been fitted to both ends of the rod-like ferrite core 12. The accommodation of the transmitting ferrite antenna 10 into the antenna accommodation concave part 14 is performed by fitting the cap-like antenna frame 16 mounted to the both ends of the rod-like ferrite core 12 to antenna frame fitting parts 15 formed in both ends of the antenna accommodation concave part 14. By doing like this, the transmitting ferrite antenna 10 is firmly held to the transmitter case 1 and protected from the impact applied to the transmitter case 1.

As mentioned above, although the transmitting ferrite antenna 10 is held in the transmitter case 1 by fitting the cap-like antenna frame 16 to the antenna frame fitting parts 15 in the both ends of the antenna accommodation concave part 14, there is adopted means for making the holding of the transmitting ferrite antenna 10 into the transmitter case 1 additionally strong. That is, as shown in FIG. 3, a convex part 2a is formed in one part of the case back 2 of the transmitter case 1. The convex parts 2a are formed in positions, of the case back 2, corresponding to both end parts of the antenna accommodation concave part 14 of the transmitter case 1. Accordingly, as to the transmitting ferrite antenna 10 having been accommodated in the antenna accommodation concave part 14 with the cap-like antenna frame 16 being fitted to the antenna frame fitting parts 15 in the both ends of the antenna accommodation concave part 14, when the case back 2 of the transmitter case 1 is closed, the cap-like antenna frame 16 is pressed by the convex part 2a of the case back 2. By doing like this the transmitting ferrite antenna 10 is strongly held in the transmitter case 1 and protected from the impact applied to the transmitter case 1.

Further, the means for making the holding of the transmitting ferrite antenna 10 into the transmitter case 1 additionally strong is, as shown in FIG. 4, to bond the transmitting ferrite antenna 10 to the antenna accommodation concave part 14 by an adhesive 17 having the elasticity. That is, the antenna accommodation concave part 14 is the groove of the shape and the dimension, by which to its both ends there is fitted the cap-like antenna frame, and is the groove in which its intermediate part accommodates the winding 11 of the ferrite antenna 10 so as not to contact with the wall face. For this reason, an interstice is generated between an inner wall of the antenna accommodation concave part 14 and the transmitting ferrite antenna 10, especially between the former and an outer peripheral face of the winding 11. To this interstice, there is filled the adhesive 17 having the elasticity. By the filled adhesive 17 having the elasticity, the transmitting ferrite antenna 10 is bonded to the antenna accommodation concave part 14. By doing like this, the transmitting ferrite antenna 10 is strongly held in the transmitter case 1 and protected from the impact applied to the transmitter case 1.

By the present invention, it has been made possible to accommodate and fix the ferrite antenna in and to the case without using the complicated fixing structure and while being adapted so as not to undergo the influence of the external impact. That is, it follows that the ferrite antenna is disposed in the case without directly contacting with the case and, since the impact is not transmitted to the ferrite antenna even if the case undergoes the impact such as dropping, it has become possible to prevent a breakdown accident of the antenna.

What is claimed is:

1. A heartbeat measuring device for measuring a heart rate of a user comprising:
a ferrite antenna;
a cap-like antenna frame;
wherein the ferrite antenna has been accommodated in an antenna accommodation concave part of a case through the cap-like antenna frame having an elasticity.

2. A heartbeat measuring device according to claim 1, wherein the antenna frame is a cap-like antenna frame mounted to both ends of a rod-like ferrite core of the ferrite antenna.

3. A heartbeat measuring device according to claim 1, wherein the ferrite antenna in which the cap-like antenna frame has been mounted to both ends is held in the antenna accommodation concave part with the cap-like antenna frame being fitted respectively to both ends of the antenna accommodation concave part of the case.

4. A heartbeat measuring device according to claim 1, wherein the ferrite antenna in which the cap-like antenna frame has been mounted to both ends is held in the antenna accommodation concave part with the cap-like antenna frame being pressed by a convex part formed in a case back of the case.

5. A heartbeat measuring device according to claim 3, wherein an adhesive having the elasticity is filled in an interstice between the antenna accommodation concave part and the ferrite antenna accommodated in the former.

6. A heartbeat measuring device according to claim 4, wherein an adhesive having the elasticity is filled in an interstice between the antenna accommodation concave part and the ferrite antenna accommodated in the former.

7. A heartbeat measuring device according to claim 1, wherein the ferrite antenna is used in a transmitter for transmitting the heart rate of the user while being mounted to a chest of the user.
